(19) United States
(12) Patent Application Publication
(10) Pub. No.: US 2021/0275668 A1
(43) Pub. Date: Sep. 9, 2021

(54) COMBINATION OF FACTOR VII AND AN ANTI-FACTOR IX/X BISPECIFIC ANTIBODY

(71) Applicant: Laboratoire Français du Fractionnnement et des Biotechnologies, Les Ulis (FR)

(72) Inventor: Jean-Luc Plantier, Croix (FR)

(73) Assignee: Laboratoire Français du Fractionnnement et des Biotechnologies, Les Ulis (FR)

(21) Appl. No.: 17/251,695

(22) PCT Filed: Jun. 14, 2019
(86) PCT No.: PCT/FR2019/051456

§ 371 Date: Dec. 11, 2020

(30) Foreign Application Priority Data
Jun. 14, 2018 (FR) 1855239

(51) Int. Cl. A61K 39/395; A61K 38/48; C07K 16/36; A61P 7/04

(52) U.S. Cl. CPC A61K 39/3955; A61K 38/4846; C07K 16/36; A61K 2039/545; C07K 2317/31; C07K 2317/24; A61P 7/04

(57) ABSTRACT

The invention concerns a combination comprising transgenic factor VII and a multispecific antibody directed against factor IX and X, for simultaneous or separate administration.

The invention concerns pharmaceutical compositions useful for the treatment of a coagulation disorder, such as hemophilia A, particularly in a patient with type A hemophilia with the development of factor VIII inhibitor antibodies.

### TECHNOLOGICAL BACKGROUND

Coagulation involves two pathways, one intrinsic and the other extrinsic, leading to a final common pathway. The combination of both mechanisms ensures the formation of a solid and flexible blood clot that resists blood pressure. Via the action of thrombin, fibrinogen undergoes chemical modifications that lead to the formation of fibrin. Fibrin is necessary to the formation of a clot.

The intrinsic pathway comprises factors present in the bloodstream and the coagulation process starts within the blood vessel itself. The extrinsic pathway involves tissue factors not normally present in the bloodstream and that are released during a vascular injury. Factor VII is a glycoprotein that is involved in the extrinsic coagulation pathway. In order to initiate the coagulation cascade, FVII must be activated into FVIIa. Once activated, FVIIa complexes with the tissue factor (TF) protein associated with two phospholipids, which is released during vascular injury. FVIIa alone (not complexed with tissue factor) exhibits low proteolytic activity. The FVIIa-FT complex then transforms factor X into factor Xa in the presence of calcium ions. This complex also acts on the activation of factor FIX into FIXa, thereby catalyzing the intrinsic pathway. Factors IXa and Xa, in turn, activate activated factor VII.

Factor IX and factor X are involved in the intrinsic coagulation pathway. Activated factor IX enables factor X to be activated into factor Xa.

Factor Xa complexed with activated factor FV and prothrombinase transforms prothrombin into thrombin. Thrombin then acts on fibrinogen to transform it into fibrin and also enables FVIII and FV to be activated into FVIIIa and FVa, respectively. Prothrombin, for its part, permits activating factor XIII into FXIIIa, responsible for the consolidation of the fibrin clot, in the presence of calcium naturally present in the plasma.

Nevertheless, when a coagulation factor is lacking, the coagulation cascade is interrupted or deficient and then we speak of abnormal coagulation.

Activated factor VII acts locally in the presence of tissue factor released after tissue injury engendering bleeding, even in the absence of factor VIII or IX. This is why factor VII, preferably in the activated form, is used for the treatment of certain blood coagulation disorders that present as bleeding.

Factor VII is thus used in treating patients with hemophilia, presenting a deficiency in factor VIII (type A hemophilia) or in factor IX (type B hemophilia), as well as patients presenting other coagulation factor deficiencies, for example, a hereditary deficit in FVII. FVII is also recommended in stroke treatment.

Some hemophilia patients develop antibodies that inhibit the factor VIII administered, generally in the concentrated form, as hemophilia treatment. This is currently the most common complication of hemophilia treatment.

Bispecific antibodies targeting FIX or FIXa and FX or FXa, such as emicizumab, are used to treat hemophilia A patients with anti-factor VIII antibodies. These antibodies functionally replace FVIII by promoting the activation of FX by FIXa by bringing these two molecules together. These antibodies have a long-lasting effect.

The combination of recombinant FVIIa from cell culture (such as Novoseven®, produced in BHK cells) with emicizumab (such as ACE910 or Hemlibra®) has been tested (R. HARTMANN, et al. OR36|Synergistic Effects of a Procoagulant Bispecific Antibody and FEIBA or Factor VIIA on Thrombin Generation (Haemophilia (2017), 23 (Suppl. 2), 11-27)). This combination only showed an additive effect on the treatment of coagulation disorders.

Consequently, there is a need for a pharmaceutical combination that permits better management of hemophilia A patients, and more particularly patients with anti-factor VIII

### SUMMARY OF THE INVENTION

The invention proposes combining transgenic factor VII with a multispecific antibody directed against factors IX and X.

According to the invention, the combination of the invention of factor VII obtained by transgenesis and antibodies directed against factor IX and factor X induces a synergistic effect in the treatment of coagulation disorders, and particularly for the treatment of patients with hemophilia A with anti-FVIII inhibitors and patients deficient in FVII.

One aspect of the invention is therefore a pharmaceutical composition comprising:

a. transgenic factor VII, and

b. a multispecific antibody, preferably bispecific, directed against factor IX and factor X, such as, for example emicizumab.

Preferably, factor VII is in the form of activated factor VII (FVIIa).

In a particular embodiment, factor IX is in the form of activated factor IX (FIXa) and/or factor X is in the form of activated factor X (FXa).

Preferably, said transgenic factor VII is a human factor VII derived from production by epithelial cells of the mammary glands of a non-human transgenic mammal, for example a rabbit transgenic for human factor VII.

The invention also provides a combination product comprising a. transgenic factor VII, and b. a multispecific antibody directed against factor IX and factor X, for its use in the prevention or treatment of a coagulation disorder, such as hemophilia A, more particularly hemophilia A with factor VIII inhibitors (FVIII).

Preferably, the combination product is in the form of a pharmaceutical composition that comprises both transgenic factor VII and said antibody.

Alternatively, transgenic factor VII and the antibody are in the form of separate compositions, suitable for simultaneous or separate (for example sequential) administration to the patient.

Another object of the also invention relates to a kit comprising:
A container containing transgenic factor FVII; and
Another container containing an antibody directed against factor IX and factor X.

### LIST OF FIGURES

FIG. 1: Assessment of the synergic thrombogenic effect of the Sevenfact™+Hemlibra® combination on batch 1 of Hemophiliac A plasma after induction with TF/PL. (A) Assessment of the synergic thrombogenic effect on ETP, (B) Assessment of the synergic thrombogenic effect on the thrombin generation peak, (C) Assessment of the synergic thrombogenic effect on velocity.

FIG. 2: Assessment of the synergic thrombogenic effect of the Sevenfact™+Hemlibra® combination on batch 2 of Hemophiliac A plasma after induction with TF/PL. (A) Assessment of the synergic thrombogenic effect on ETP, (B) Assessment of the synergic thrombogenic effect on the thrombin generation peak, (C) Assessment of the synergic thrombogenic effect on velocity.

### DETAILED DESCRIPTION OF THE INVENTION

### General Definitions

The coagulation phenomenon consists of a cascade of enzymatic reactions involving coagulation factors present in the form of proenzymes that, in the presence of certain cofactors, are converted by proteolytic cleavage into their “activated” form. The activated form of each factor present in the form of inactive precursor is designated by the letter a. Thus, FVIIa results, in vivo, from the cleavage of zymogen by various proteases (FIXa, FXa, FVIIa) into two chains joined by a disulfide bridge.

The term “treatment” or “treat” generally designates improvement, prophylaxis, or reversal of a disease or disorder, or at least a symptom, for example, slowing the progression of a disease or stabilizing a symptom. Delay of onset of a disease or disorder, or at least a symptom, is also included.

The term “prevention” or “prevent” designates a reduction in the risk of developing or acquiring a specific disease or disorder.

In the present invention, “patient” or “subject” means any mammal, and more particularly human beings, male or female, of any age, including children.

The term “pharmaceutical composition” refers to preparations permitting the biological activity of active ingredients and not containing any additional component toxic for the subjects to whom the composition is administered.

### Transgenic Factor VII

The term “Factor VII” or “FVII” includes polypeptides comprising sequence 1-406 of wildtype human factor VII (as described in U.S. Pat. No. 4,784,950) or FVII derived from another species (for example, bovine, porcine, canine, murine). It also comprises natural allelic variations of factor VII that may exist, in any form or degree of glycosylation or other post-translational modification. Thus, the term “factor VII” also includes FVII variants that have the same or better biological activity relative to the activity of the wildtype, these variants particularly including polypeptides different from wildtype FVII by insertion, deletion or substitution of one or more amino acids.

Unless otherwise indicated, in the present description, the term “factor VII” will refer to either uncleaved FVII (zymogen) or activated factor VII (FVIIa)

FVIIa is therefore composed of a light chain of 152 amino acids of molecular weight of approximately 20 kDa and a heavy chain of 254 amino acids of molecular weight of approximately 30 kDa linked together by a single disulfide bridge (Cys135-Cys262).

“Recombinant factor VII” means any factor VII derived from genetic engineering and resulting from the expression of the corresponding gene in any microorganism, plant or transgenic plant. Microorganism means any bacterial, fungal, viral or cellular system. Recombinant factor VII can also be produced from eukaryote cells in culture, such as plant or mammal cells, for example, animal or human cells.

“Transgenic factor VII” means any recombinant factor VII obtained from an animal transgenic for factor VII.

“Transgenic animal” means any nonhuman animal with a modification in its genome intended to enable the protein of interest to be expressed (here, factor VII). The genome modification may result from an alteration, modification or insertion of a gene. This modification may be due to the altering or mutagenic agents conventionally used or even done by directed mutagenesis. Genome modification may also result from an insertion or substitution of a gene or genes in their wildtype or mutated form. The transgenic animal may be chosen, in a nonlimiting manner, from rabbit, goat, cow, camel, hamster, mouse, rat, horse, sow, dromedary, sheep or llama. In a particular embodiment, an animal that does not express a1,3-galactosyltransferase can be chosen.

The expression “biological activity of factor Vila” means the ability of FVIIa to generate thrombin, for example, on the surface of activated platelets. The activity of factor VII can be assessed in various ways. The biological activity of FVIIa can be quantified, for example, by measuring the ability of an FVII composition to promote blood clotting by using a plasma deficient in FVII and thromboplastin, as described, for example, in U.S. Pat. No. 5,997,864. In this test, biological activity is assessed relative to a control sample and is converted into “FVII units” by comparison with a pooled standard human serum containing 1 unit/mL of factor VII activity. Alternatively, the biological activity of factor VII can be quantified by (i) measuring the ability of factor Vila to produce factor Xa in a system comprising a tissue factor (TF) surrounded by a lipid membrane and factor X (Persson et al. J. Biol. Chem. 272: 19919-19924, 1997); (ii) measuring hydrolysis of factor X in an aqueous system; (iii) measuring the physical bond of FVIIa to TF by means of surface plasmon resonance (Persson, FEBS letts, 413:359-363, 1997), (iv) measuring the hydrolysis of a synthetic substrate or (v) measuring the generation of thrombin in an in-vitro system independent of TF.

In a preferred embodiment, the FVII described here is a polypeptide whose peptide sequence can be that of natural human FVII, i.e., the sequence present in humans who do not have disorders related to FVII. Such a technique is described in document EP 0 200 421. Advantageously, the FVII sequence used in the invention is SEQ ID NO: 1.

“Synergy” or “synergistic effect” means, preferably, an effect of the combination of two products that is greater than twice the sum of the effects of each of the products taken separately. According to the present invention, a synergistic effect is obtained when the use of a transgenic FVIIa in combination with a multispecific antibody directed against factor IX and factor X permits obtaining an effect greater than 2 times the sum of the effect obtained with a transgenic FVIIa alone and the effect obtained with a multispecific antibody directed against factor IX and factor X alone, on at least one thrombin generation parameter. This thrombin generation parameter is chosen from peak height, velocity or endogenous thrombin potential (ETP).

In a particular embodiment, FVIIa is administered at a concentration less than or equal to 105 nM, preferably less than 100 nM.

In a particular embodiment, the multispecific antibody directed against factor IX and factor X is administered at a concentration of less than 600 nM, preferably less than 550 nM, preferably less than 500 nM, preferably less than 450 nM, preferably less than 400 nM, preferably less than 350 nM, preferably less than 325 nM.

In a particular embodiment, factor VII is obtained from the milk of a transgenic animal.

A method of producing a recombinant protein in the milk of a transgenic animal may comprise the following steps: a synthetic DNA molecule comprising a gene coding for a protein of interest (here, for example, human FVII), this gene being under the control of a promoter of a protein naturally secreted in milk, is integrated into an embryo of a non-human mammal. The embryo is then implanted in a female mammal of the same species. Once the mammal resulting from the embryo is sufficiently developed, lactation of the mammal is induced, and the milk is then collected. The milk contains the FVII of interest secreted by the transgenic animal. One example of protein preparation in the milk of a female mammal other than a human being is given in patent application EP0527063, the teaching of which can be reprised for the production of the factor VII of the invention.

Secretion of factor VII by mammary glands, allowing it to be secreted into the milk of the transgenic mammal, involves the control of factor VII expression in a tissue-dependent manner. Such control methods are well known to the skilled person. Expression is controlled via sequences that allow expressing the protein toward a particular tissue. These are particularly the WAP, beta-casein and beta-lactoglobulin promoter sequences and signal peptide sequences; the list is not limiting.

In a preferred embodiment, factor VII according to the invention is produced in the milk of transgenic rabbits.

In a particularly advantageous manner, expression in the rabbit's mammary glands is done under the control of the beta casein promoter well known to the skilled person. In particular, a plasmid containing the beta casein promotor is fabricated by introduction of a sequence containing the beta casein gene promoter, this plasmid being created so as to be able to receive a foreign gene placed under the control of this promoter. The gene coding for human FVII is integrated and placed under the control of the beta casein promoter. The plasmid containing the promoter and the sequence coding for the protein of interest is digested by restriction enzymes to release the DNA fragment containing the beta casein promoter and the human FVII sequence. After purification, the fragments are introduced by microinjection into the male pronucleus of wildtype rabbit embryos. The embryos are then cultured before transfer into the hormonally-prepared oviduct of wildtype females. When these females give birth, the offspring is assessed by PCR to determine the transgenic animals. The number of copies of the transgene and its integrity are revealed by the Southern technique from DNA extracted from the young transgenic rabbits obtained. The concentration of human FVII expressed in the milk of female transgenic descendents is assessed via immunoenzymatic tests.

In a particular embodiment, the factor VII useful in the invention is obtained by a method comprising the following steps:

(a) insertion of a DNA sequence comprising a gene coding for factor VII into a non-human mammal embryo, said gene being under the transcriptional control of the beta casein promoter,

(b) transfer of the embryos obtained in step a) into the oviduct of non-human mammal females so that it develops into an adult non-human mammal.

(c) induction of lactation in the adult nonhuman mammal obtained in step b) of the female type or in a female descendent of this non-human mammal in which the gene and the promoter are present in its genome.

(d) collection of milk from said non-human mammal, and

(e) purification of the FVII present in the collected milk.

The FVII useful here has a substantially homogenous isoelectric point.

“Isoelectric point” or “pI” means the pH for which the net elementary charge of the factor VII or factor Vila molecule is zero, i.e., the pH at which the molecule is electrically neutral (zwitterionic form). The isoelectric point of the factor VII according to the invention can be measured by implementing a technique well known to the skilled person such as isoelectric focusing (“IEF”). This electrophoretic technique separates proteins on the basis of their isoelectric point. It consists of migration, induced by a uniform electrical current, of proteins in a pH gradient until they reach a pH equivalent to their specific isoelectric point, at which time they stop migrating since their net charge is zero. IEF gels are used to determine the isoelectric point of a given protein.

“Substantially homogenous” means that at least 90%, preferably at least 95% of the factor VII molecules of the composition have an isoelectric point comprised in a pH unit difference of less than or equal to 1.2. In another embodiment of the invention, at least 50%, preferably at least 55%, preferably 60% of the transgenic factor VII molecules of the composition have an isoelectric point comprised in a pH unit difference of less than 1, preferably of less than 0.5. In another embodiment of the invention, at least 50%, preferably at least 55%, preferably 60% of the factor VII molecules of the composition have an isoelectric point comprised in a pH unit difference of 0.4.

“N-glycan forms” means all of the N-glycan forms present at the two N-glycosylation sites of the factor VII of the invention. The N-glycan forms are called monocharged if their total charge is equal to 1. In the present invention, “charge” means a phosphate group, a sulfate group, or a sialic acid molecule. Thus, the N-glycan forms are called monocharged if they contain only one phosphate group or one sulfate group or one sialic acid molecule. As opposed to the term “monocharged”, the term “bicharged” means that the total charge carried by the N-glycan forms is equal to 2, i.e., they have two charges chosen from a phosphate group, a sulfate group and/or a sialic acid molecule. In other words, the bicharged N-glycan forms have one sialic acid molecule and one phosphate group, or one sialic acid molecule and one sulfate group, or two sialic acid molecules, or two phosphate groups, or two sulfate groups, or a phosphate group and a sulfate group. The term “tricharged” means that the total charge carried by the N-glycan forms is equal to 3, i.e., they have three charges chosen from a phosphate group, a group sulfate and/or a sialic acid molecule. In other words, tricharged N-glycan forms have one sialic acid molecule, one phosphate group and one sulfate group, or two sialic acid molecules and one phosphate group, or two sialic acid molecules and one sulfate group, or one sialic acid molecule and two phosphate groups, or one sialic acid group and two sulfate groups, or one phosphate group and two sulfate groups, or one sulfate group and two phosphate groups or three sialic acid molecules, or three phosphate groups, or three sulfate groups. The term “neutral” means that the N-glycan forms do not contain any charge. The charge of the N-glycan forms of factor VII according to the invention can be measured by implementing a method well known to the skilled person, particularly by ultra high performance liquid chromatography with an anion exchange resin coupled to detection by fluorescence (AEX-UPLC/FD). This method allows separating the different N-glycan forms according to their apparent charge (see, in particular, Hermentin et al, Glycobiology, vol. 6, no. 2, 1996). In the context of anion exchange chromatography, a positively-charged resin is used as stationary phase. These positively-charged resins are generally made up of a crosslinked polymer or gel, onto which positively charged groups are grafted. In an advantageous embodiment of the invention, an aminopropyl-type weak anion exchange column is used.

In the case of the factor VII composition according to the invention, it appears that among all the N-glycan forms of the factor VII of the composition, at least 50% of the N-glycan forms, at least 60% of the N-glycan forms, preferably at least 65%, preferably at least 70%, preferably at least 75%, preferably at least 80%, preferably at least 85%, preferably at least 90%, preferably at least 95%, are monocharged. In a preferred embodiment, the factor VII molecules having monocharged N-glycan forms represent between 50% and 95% of the factor VII molecules of the composition, preferably between 50% and 90% of the factor VII molecules of the composition, preferably between 50% and 80% of the factor VII molecules of the composition, preferably between 50% and 75% of the factor VII molecules of the composition, preferably between 50% and 70% of the factor VII molecules of the composition, preferably between 50 and 65% of the factor VII molecules of the composition, preferably between 50% and 60% of the factor VII molecules of the composition.

The substantially homogenous isoelectric point of the factor VII composition of the combination according to the invention results from the combination of the glycosylation and γ-carboxylation properties of the FVII molecules that compose it.

The transgenic factor VII useful here has characteristics of post-translational modifications. In particular, these are glycosylation modifications, such as two N-glycosylation sites with a zero or very low amount of Galα1,3Gal in the FVII composition, or still low enough not to be immunogenic. In contrast, the FVII described here is not a plasma FVII, i.e., it is not a purified product from human or animal plasma. More particularly, the transgenic FVII useful here has post-translational modifications, as well as two O-glycosylation with defined glycan units, a γ-carboxylation, and specific disulfide bridges.

The FVIIa useful here can include several post-translational modifications: the first nine or ten N-terminal glutamic acids are γ-carboxylated, Asp₆₃ is partially hydroxylated, Ser₅₂ and Ser₆₀ are O-glycosylated and respectively bear glucose (xylose)₀₋₂ and fucose units, Asn₁₄₅ and Asn₃₂₂ are N-glycosylated predominantly by monosialylated biantennary complex structures. Advantageously, at least 80% of the transgenic factor VII molecules useful here have a γ-carboxylation on nine glutamic acid residues. In another embodiment, at least 85% of said molecules have a γ-carboxylation on nine glutamic acid residues. In another embodiment, between 85% and 100%, preferably between 90% and 100%, preferably between 95% and 100% of said molecules have a γ-carboxylation on nine glutamic acid residues. Advantageously, the degree of γ-carboxylation on glutamic acid residue 35 (Glu 35) of the factor VII molecules of the composition is less than 20%. In another embodiment, the degree of γ-carboxylation of residue Glu35 is less than 15%, preferably less than 10%, preferably less than 5%.

The Galα1,3Gal unit is a structure composed of two galactoses linked at a1,3. It is located at the end of the oligosaccharide antennas of the N-linked structures. This unit is known for its immunogenicity. Therefore, it is preferred to produce FVII or FVIIa whose number of structures Galα1,3Gal is zero or so low that it cannot be distinguished from the background noise obtained by the measurements implemented by currently available analysis devices. This expression equivalently designates all transgenic FVII whose amount of Galα1,3Gal is close to that of plasma FVII. Advantageously, the amount of Galα1,3Gal of the FVII composition described here is not immunogenic for humans. Furthermore, the FVII useful here preferably comprises two N-glycosylation sites, in position 145 and 322, and two O-glycosylation sites, in position 52 and 60, like human FVII. In an N-glycosylation site, the oligosaccharide chains are linked to an asparagine (N-linked). In an O-glycosylation site, the oligosaccharide chains are linked to a serine. The units linked to these amino acids will be different for each protein of the composition. However, for the entire composition, the amount of each glycan unit or even each sugar can be quantified.

The percentages of different glycans given in the present application do not take 0-glycosylation into account.

Preferably, the FVII composition is characterized in that, among all the glycan units of the FVII of the composition, at least 40% are monosialylated biantennary glycan forms. In another embodiment, the monosialylated biantennary forms are present in at least 50%. In another embodiment, the monosialylated biantennary forms are present in at least 60%, preferably at least 65%, preferably at least 70%.

Advantageously, the monosialylated biantennary glycan forms of FVII are predominant. The FVII composition is characterized in that at least some of the factor VII sialic acids involve a2-6 bonds.

Advantageously, at least 65% of the sialic acids of FVII involve a2,6 bonds. Very advantageously, at least 70%, even 80% and, in particular, at least 90% of the FVII sialic acids involve a2,6 bonds.

In a particularly preferred way, all the sialic acids involve a2,6 bonds, i.e., all the sialic acids are bound to galactose by an a2,6 bond. The FVII composition described here can also comprise sialic acids with a2-3 bonds.

According to embodiments of the invention, 65% to 100% of the FVII sialic acids involve a2,6 bonds. More preferably, 70% or 80% to 100% of the FVII sialic acids involve a2,6 bonds.

Advantageously, among the monosialylated biantennary glycan forms of FVII, the predominant glycan forms are nonfucosylated.

Preferably, these nonfucosylated monosialylated biantennary glycan forms are present in the FVII of the composition in an amount greater than 20%. Advantageously, this amount is greater than 25%, or greater than 40%. In a particularly advantageous manner, the degree of fucosylation of the FVII composition is comprised between 20% and 50%. In one embodiment, this degree can be less than 20%.

In a particular embodiment, at least 10%, preferably at least 15%, preferably at least 20%, preferably at least 25% of the N-glycan forms of the factor FVII of the composition are high mannose/hybrid.

Preferably, the glycosylation profile described here procures improved biological activity and stability for FVII. Factor VII compositions with a substantially homogenous isoelectric point facilitate the formulation step at an optimal pH, preferably at an optimal pH of 6.0±0.2, of pharmaceutical composition by preventing precipitation of FVII. Indeed, it is known that at the isoelectric point of a molecule, these will have a tendency to aggregate and precipitate. The factor VII molecules used in the composition of the invention have an isoelectric point comprised between 6.6 and 7.0. This results in a better stability of the factor VII composition, especially when it is formulated at a pH below the isoelectric point, and in particular at pH 6.0 The improvement in the stability of the factor VII composition prevents the electrostatic interactions responsible for soluble and insoluble precipitation and aggregation phenomena, as well as preventing the loss of raw materials and therefore a drop in yield resulting in a loss of the quantity of active principle and therefore potentially in a loss of activity.

In a preferred embodiment, the transgenic FVII is produced by the rabbit in its milk, allowing a composition to be obtained where each factor VII molecule of the composition has two N-glycosylation sites. Preferably all the FVII molecules of the composition have an amount of Galα1,3Gal glycan units less than 4%, or even none. Thus, advantageously, the transgenic FVII produced by the rabbit does not have a Galα1,3Gal unit.

FVII can be purified from milk by techniques known to the skilled person. For example, a method of purifying a protein of interest from milk, as described in U.S. Pat. No. 6,268,487, can comprise the following steps consisting of: a) subjecting the milk to tangential filtration through a membrane of sufficient porosity to form a retentate and a permeate, the permeate containing the exogenous protein, b) subjecting the permeate to a chromatographic capture device so as to displace the exogenous protein and obtain an effluent, c) combining the effluent and the retentate, d) repeating steps a) to c) until FVII is separated from lipids and casein micelles, and the FVII is recovered.

Advantageously, the FVII of the invention is in the activated form. In one embodiment, FVII can be activated in vitro by factors Xa, Vila, IIa, IXa or XIIa. FVII can also typically be activated during its purification process, in particular by passage through positively charged chromatography columns.

### Multispecific Antibody

“Multispecific antibody” means any antibody possessing at least two binding sites specific to at least two different antigens, or different epitopes of the same antigen. The term “specific” means that the antibody has the ability to recognize and bind an antigen, substantially without cross reaction with another antigen. Advantageously, the antibody has an affinity constant kD relative to each antigen of at least 10⁻⁶ M, preferably at least 10⁻⁷ M, more preferably at least 10⁻⁸ M, 10⁻⁹ M, or 10⁻¹⁰ M.

Thus, the antibodies useful in the invention have the ability to specifically bind both to coagulation factor IX and to coagulation factor X in the activated or non-activated form.

The antibody used in the invention, which has the ability to specifically bind both coagulation factor IX and coagulation factor X preferentially has the ability to work as a substitute for factor VIII (FVIII), which means that this antibody promotes the activation of FX by FIXa.

Such multispecific, preferably bispecific, antibodies can be obtained by various methods known to the skilled person, for example by chemical conjugation, or by using quadromas, which result from the fusion between two hybridomas producing two different monoclonal antibodies; or even by genetic recombination.

Polynucleotides coding for such antibodies can therefore be inserted into expression vectors and expressed in host cells or organisms adapted by techniques well known to the skilled person.

Antibodies useful here can be of very simple format, constructed from single-chain Fv fragments (scFv), from two or more antibodies, associated by a suitable peptide linker.

“Fv” means the smallest antibody fragment conserving the properties of recognizing and binding the antigen. An “Fv” fragment is a dimer (V_{H}+V_{L} dimer) consisting of a variable region (V_{H}) borne by a heavy chain (H) and an adjacent variable region (V_{L}) borne by a light chain (L). Alternatively, it can be a full length antibody, preferably containing an Fc region. Several formats are possible. For example, in a first format, scFv fragments of an antibody A are fused to the ends (generally N-terminus) of the heavy chains of an antibody B. The resulting antibody has a single type of heavy chain, which contains the V_{H}, CH1, CH2 and CH3 domains of antibody B and the V_{H} and V_{L} domains of antibody A, and a single type of light chain that contains the V_{L} and CL domains of antibody B (Qu et al. Blood, 111, 2211-2219, 2008). In a second format, the heavy chain and the light chain of an antibody A are associated with the heavy chain and light chain of an antibody B. Where appropriate, mutations, for example “knobs into holes” (Ridgway et al, Protein Eng, 9, 617-21, 1996; U.S. Pat. No. 7,695,936) can be introduced to prevent mismatches.

Unless indicated to the contrary, in the present description, the term “factor IX” will refer to either inactivated factor IX or activated factor IX (FIXa).

Unless indicated to the contrary, in the present description, the term “factor X” will refer to either inactivated factor X or activated factor X (FXa).

An antibody recognizing (i) FIX and/or FIXa, and (ii) FX and/or FXa can be obtained, in particular, according to the methods described in patent applications WO2005/035756, WO2006/109592 or WO2012/067176.

In a preferred embodiment, said antibody is emicizumab. The production of this antibody is described, for example, in patent application WO2018047813 or patent application EP1688488.

### Pharmaceutical Composition and Dosages

Factor VII and antibodies can be formulated in the form of separate pharmaceutical compositions, or combined within the same pharmaceutical composition.

In the case of separate administration, the FVII and antibodies can be formulated in a manner suited to administration via different routes or the same route.

Thus, for example, FVII can be administered intravenously, subcutaneously or intramuscularly.

The antibody can also be administered, for example, intravenously, subcutaneously or intramuscularly.

A factor VII composition can be, for example, like the one described in patent application WO2010/149907

Thus, in one example of embodiment, the composition comprises:
factor VII, preferably in the factor Vila form;
arginine, possibly in the hydrochloride form;
isoleucine;
lysine;
glycine;
trisodium citrate or calcium chloride;
and, where appropriate, polysorbate 80 or polysorbate 20.

More particularly, the composition can comprise:
factor VII, preferably in the factor Vila form;
10 to 40 g/L of arginine, possibly in the hydrochloride form;
4.2 to 6.6 g/L of isoleucine;
0.6 to 1.8 g/L of lysine;
0.6 to 1.8 g/L of glycine;
0 to 0.2 g/L of trisodium citrate or 1 to 2 g/L of calcium chloride;
and, where appropriate, 0 to 0.5 g/L of polysorbate 80.

The FVII composition, which optionally also comprises at least one multispecific antibody as described here, can be stored in the liquid for or the solid form, typically obtained by desiccation. The compositions disclosed above are determined relative to compositions in the liquid form, before desiccation, or after reconstitution in the form of preparation for injection.

Desiccation is a method for high-stage water elimination. It is dehydration aiming to eliminate as much water as possible. This phenomenon can be natural or forced. This desiccation can be conducted by means of freeze drying, spray drying and spray-freeze drying.

The preferred method for obtaining the solid form of the composition for pharmaceutical usage described here is freeze drying.

Freeze-drying methods are well known to the skilled person, see, for example [Wang et al, Lyophilization and development of solid protein pharmaceuticals, International Journal of Pharmaceutics, Vol 203, p 1-60, 2000].

Other appropriate processes for reducing the degree of humidity or the water content of the composition can be envisaged. Preferably, the degree of humidity is less than or equal to 3% by weight, preferably less than or equal to 2.5%, preferably less than or equal to 2%, preferably less than or equal to 1.5%.

The solid composition, preferably in the freeze-dried form, can be dissolved in water for injection (WFI), to obtain a formulation for therapeutic usage.

The injectable formulation can be administered parenterally (intravenously, subcutaneously, intramuscularly), in a quantity assessed by the practitioner. The administration of the liquid form (before desiccation) or the solid form, by any route and any means appropriate, is not excluded.

The FVII dosage useful in the invention can be determined in an appropriate way depending on the type of formulation, administration method, patient age and weight, patient symptoms, severity of the disease, etc.

The FVII dose to administer according to the invention can be chosen between 270 μg/kg and 2.70 μg/kg. Preferably, the dose of FVII to be administered is less than 270 μg/kg of bodyweight, preferably it is less than 225 μg/kg of bodyweight, preferably it is less than 180 μg/kg of bodyweight, preferably it is less than 135 μg/kg of bodyweight, preferably it is less than 90 μg/kg of bodyweight, preferably it is less than 45 μg/kg of bodyweight, preferably it is less than 9 μg/kg, preferably it is less than 5.4 μg/kg, preferably it is less than 2.7 μg/kg.

A multispecific antibody composition, such as emicizumab antibody, is, for example, like the ones described in patent applications WO2017/188356 and WO2018/047813.

Thus, in one example of embodiment, the composition is a liquid composition.

In one example of embodiment, the composition comprises:
antibodies bispecific for factor IX and factor X
a surfactant such as poloxamer 188 or polysorbate 20
histidine-aspartic acid buffer
arginine

More particularly, the composition can comprise:
20 mg/mL to 180 mg/mL of antibodies bispecific for factor IX and factor X,
0.2 mg/mL to 1 mg/mL of poloxamer 188,
10 mM to 40 mM of histidine-aspartic acid buffer
100 mM to 300 mM of arginine,

at a pH comprised between 4.5 and 6.5

The dosage of the multispecific antibody composition, such as emicizumab antibody, useful in the invention, can be appropriately determined depending on type of formulation, method of administration, patient age and the weight, patient symptoms, severity of the disease, etc. The antibody dose can be, for example, 0.3 to 5 mg/kg, preferably at most 3 mg/kg once a week during an initiation period, which can last 4 weeks, for example, followed by a maintenance dose, which is preferably lower, for example, 1.5 mg/kg once a week. Preferably, the dose of antibody administered is less than 5 mg/kg of bodyweight, preferably it is less than 3 mg/kg of bodyweight, preferably it is less than 1.5 mg/kg of bodyweight, preferably it is less than 1 mg/kg of bodyweight, preferably it is less than preferably it is less than 0.5 mg/kg of bodyweight, preferably it is less than 0.1 mg/kg of bodyweight, preferably it is less than 0.05 mg/kg of bodyweight.

The antibody composition useful in the invention can be administered to a patient via any appropriate route, for example intravenously, intramuscularly, intraperitoneally, intra-cerebrospinally, transdermally, subcutaneously, intra-articularly, sublingually, intrasynovially, orally or by inhalation. Preferably, the intravenous route or subcutaneous route is favored.

According to a particular embodiment, the factor VII and antibody are administered to the patient simultaneously.

According to another particular embodiment, the factor VII and antibody are administered to the patient separately, preferably sequentially.

### Therapeutic Indications

The combination described here prevents or treats coagulation disorders, in particular hemophilia presenting a factor VIII deficit (type A hemophilia, preferably acquired type A hemophilia).

Preferably, the patients are patients who have type A hemophilia, with anti-factor VIII.

The combination described here prevents or treats coagulation disorders, in particular factor VII deficiencies.

The combination described here combines the rapid effect of FVII activating the extrinsic pathway of the coagulation cascade and the prolonged effect of the multispecific antibodies described here that activate the intrinsic pathway of the coagulation cascade. The combination makes it possible to offer better patient management.

### EXAMPLES

### Example 1: Purification and Extraction of Transgenic FVII

The factor VII purification and extraction method implemented in this example is the one described in application EP12305882. The steps of this method are described below. Transgenic rabbit milk is obtained from the transgenic rabbit line. Frozen milk from transgenic rabbits is thawed and concentrated in the form of a pool of transgenic rabbit milk.

The pool of transgenic rabbit milk thus obtained is then submitted to clarification step using a depth filter with a porosity of 0.2 μm, in order to remove lipids and insoluble compounds. The milk thus clarified is then subjected to a viral inactivation step by a detergent solvent, for example, polysorbate 80 or tri-n-butyl phosphate at 25° C.±2° C. for at least two hours. Such a treatment effectively inactivates viruses, and, in particular, non-enveloped viruses. The clarified and virally-inactivated milk is then subjected to an affinity chromatography step using an affinity ligand specific for factor VII/factor Vila. The factor VII eluate obtained from this chromatography step is then subjected to an ultrafiltration and formulation step, thus making it possible to obtain an intermediate factor VII concentrate with a purity of 95%.

The intermediate factor VII concentrate is then subjected to a filtration step using a filter with a porosity of 0.1 μm to 0.2 μm followed by a nanofiltration step through filters with a porosity of 20 nm then 15 nm. The product thus obtained and containing factor VII is then subjected to Q Sepharose XL gel chromatography then a step of CHT-I chromatography followed by Superdex 200 SEC chromatography. The factor VII concentrate thus obtained is then subjected to a stabilization step then filtration through a filter with a porosity of 0.2 μm.

The method thus described makes it possible to obtain a factor VII concentrate having a purity of approximately 99.9995%.

### Example 2: Comparison of the Thrombogenic Potential of Novoseven®, Sevenfact® and Hemlibra®

The skilled person can measure the thrombogenic potential of Novoseven®, Sevenfact® and Hemlibra® (also called emicizumab) by performing the following protocol.

Reagents:
thrombin calibrator (Stago)
5 pM PPP reagent (Stago)
PPP reagent LOW (Stago)
CK-Prest (Stago)
Fluo-buffer (Stago)
Fluo-substrate (Stago)
FVIII-deficient plasma (Siemens)
Sevenfact®/transgenic factor VII produced in rabbits 1 mg/ml (LFB)
PNP (Cryopep)
Novoseven® (NovoNordisk)
Hemlibra®/Emicizumab (Roche/Genentech/Chugaï)

### Method:

The thrombin generation test consists of activating coagulation ex vivo either with a mixture of tissue factor and phospholipids (TF/PL), or by using cephalin and then by measuring the concentration of thrombin generated over time.
Measuring the thrombogenic potential of Novoseven® after induction of coagulation with TF/P:

The thrombin generation test is performed in 80 μL of an FVIII-deficient plasma pool that mimics a hemophilia A plasma in the presence of 20 μL of PPP reagent (Stago) containing 0.5 pM of tissue factor (TF) and 4 pM of phospholipids (PL). The reaction is initiated by the addition of 20 μL of Fluca Kit (substrate+CaCl₂)) which is the start of the measurement of thrombin generation.

The therapeutic dose of FVIIa is 270 μg/kg, which corresponds to 6 μg/mL of FVIIa in the plasma, considering a recovery of 100%. The thrombin generation test is then conducted at Novoseven® doses of 0 μg/mL, 1 μg/mL, 2 μg/mL, 3 μg/mL, 4 μg/mL, 5 μg/mL, and 6 μg/mL, in the presence of 0.5 pM TF/2 pM PL (coagulation inducer).
Measuring the thrombogenic potential of Novoseven® after induction of coagulation with cephalin:

The thrombin generation test is performed in 80 μL of an FVIII-deficient plasma pool that mimics a hemophilia A plasma in the presence of 20 μL of cephalin (CK-Prest reconstituted with 5 mL of distilled H₂O).

The reaction is initiated by the addition of 20 μL of Fluca Kit (substrate+CaCl₂)) which is the start of the measurement of thrombin generation.

The thrombin generation test is conducted at Novoseven® doses of 0 μg/mL, 1 μg/mL, 2 μg/mL, 3 μg/mL, 4 μg/mL, 5 μg/mL, and 6 μg/mL, in the presence of 20 μL of cephalin (coagulation inducer).
Measuring the thrombogenic potential of Sevenfact® after induction of coagulation with TF/PL:

The thrombin generation test is performed in 80 μL of an FVIII-deficient plasma pool that mimics a hemophilia A plasma in the presence of 20 μL of PPP reagent (Stago) containing 0.5 pM of tissue factor (TF) and 4 pM of phospholipids (PL).

The reaction is initiated by the addition of 20 μL of Fluca Kit (substrate+CaCl₂)) which is the start of the measurement of thrombin generation.

The thrombin generation test is then conducted at Sevenfact® doses of 0 μg/mL, 1 μg/mL, 2 μg/mL, 3 μg/mL, 4 μg/mL, 5 μg/mL, and 6 μg/mL, in the presence of 0.5 pM TF/2 pM PL (coagulation inducer).
Measuring the thrombogenic potential of Sevenfact® after induction of coagulation with cephalin:

The thrombin generation test is performed in 80 μL of an FVIII-deficient plasma pool that mimics a hemophilia A plasma in the presence of 20 μL of cephalin (CK-Prest reconstituted with 5 mL of distilled H₂O).

The reaction is initiated by the addition of 20 μL of Fluca Kit (substrate+CaCl₂)) which is the start of the measurement of thrombin generation.

The thrombin generation test is conducted at Sevenfact® doses of 0 μg/mL, 1 μg/mL, 2 μg/mL, 3 μg/mL, 4 μg/mL, 5 μg/mL, and 6 μg/mL, in the presence of 20 μL of cephalin.
Measuring the thrombogenic potential of Hemlibra® after induction of coagulation with TF/PL:

The thrombin generation test is performed in 80 μL of an FVIII-deficient plasma pool that mimics a hemophilia A plasma in the presence of 20 μL of PPP reagent (Stago) containing 0.5 pM of tissue factor (TF) and 4 pM of phospholipids (PL).

The reaction is initiated by the addition of 20 μL of Fluca Kit (substrate+CaCl₂)) which is the start of the measurement of thrombin generation.

Hemlibra® (Roche/Genentech/Chugaï, USA), a bispecific antibody mimicking the function of FVIII, is used at the maximum concentration of 50 μg/mL, which is the concentration detected in patients on treatment (Oldenburg et al. NEJM, 2017). The thrombin generation test is then conducted at Hemlibra® doses of 0 μg/mL, 10 μg/mL, 20 μg/mL, 30 μg/mL, 40 μg/mL and 50 μg/mL, in the presence of 0.5 pM TF/4 pM PL (coagulation inducer).
Measuring the thrombogenic potential of Hemlibra® after induction of coagulation with cephalin:

The thrombin generation test is performed in 80 μL of an FVIII-deficient plasma pool that mimics a hemophilia A plasma in the presence of 20 μL of cephalin (CK-Prest reconstituted with 5 mL of distilled H₂O).

The reaction is initiated by the addition of 20 μL of Fluca Kit (substrate+CaCl₂)) which is the start of the measurement of thrombin generation.

The thrombin generation test is then conducted at Helibra® doses of 0 μg/mL, 10 μg/mL, 20 μg/mL, 30 μg/mL, 40 μg/mL and 50 μg/mL, in the presence of 20 μL of cephalin.

For all of these tests, the appearance of fluorescence is measured on a Fluoroskan Ascent fluorometer (ThermoLabsystems) at an excitation wavelength of 390 nm and an emission wavelength of 460 nm. Thrombinograms (curves showing the intensity of fluorescence over time) are then analyzed by using Thrombinoscope™ software, which converts the fluorescence value into nM of thrombin by a comparative calculation.

Thrombin is generated and the key variables to assess the potency of different medicinal products are recorded and compared: endogenous thrombin potential (ETP), peak height, latency and velocity.

### Example 3: Assessment of the Synergistic Thrombogenic Potentials of Novoseven® and Hemlibra® or SevenFact® and Hemlibra®

The skilled person can measure the thrombogenic potential of combinations of Novoseven®/Hemlibra® and Sevenfact®/Hemlibra® by performing the following protocol.

### Reagents:

The reagents, device and experimental protocol in FVIII-deficient plasma are identical to those described in Example 2.

### Method:

Measuring the thrombogenic potential of the Novoseven®+Hemlibra® combination after induction of coagulation with TF/PL:

The thrombin generation test is performed in 80 μL of an FVIII-deficient plasma pool that mimics a hemophilia A plasma in the presence of 20 μL of PPP reagent (Stago) containing 0.5 pM of tissue factor (TF) and 4 pM of phospholipids (PL).

The reaction is initiated by the addition of 20 μL of Fluca Kit (substrate+CaCl₂)) which is the start of the measurement of thrombin generation.

The thrombin generation test is conducted in the presence of 0.5 pM TF/4 pM PL (coagulation inducer) in several Novoseven®/Hemlibra® combinations. The composition containing the largest quantity of product is made up of 6 μg/mL of Novoseven® and 50 μg/mL of Hemlibra®, at their maximum.

The thrombogenic potential obtained in the presence of the combination of products is compared to the potential of the single products. To consider a synergistic effect of the product combination, lower doses are assessed to be sure not to saturate thrombin detection.

The tested compositions contain:

| Combination | Hemlibra ® (μg/mL) | NovoSeven ® (μg/mL) |
|---|---|---|
| Combination 1 | 50 | 6 |
| Combination 2 | 50 | 5 |
| Combination 3 | 40 | 4 |
| Combination 4 | 30 | 3 |
| Combination 5 | 20 | 2 |
| Combination 6 | 10 | 1 |

Measuring the thrombogenic potential of the Novoseven®+Hemlibra® combination after induction of coagulation with cephalin:

The thrombin generation test is performed in 80 μL of an FVIII-deficient plasma pool that mimics a hemophilia A plasma in the presence of 20 μL of cephalin (CK-Prest reconstituted with 5 mL of distilled H₂O).

The reaction is initiated by the addition of 20 μL of Fluca Kit (substrate+CaCl₂)) which is the start of the measurement of thrombin generation.

The thrombin generation test is conducted in the presence of 20 μL of cephalin in several Novoseven®/Hemlibra® combinations. The composition containing the largest quantity of product is made up of 6 μg/mL of Novoseven® and 50 μg/mL of Hemlibra®, at their maximum. The thrombogenic potential obtained in the presence of the combination of products is compared to the potential of the single products. To consider a synergistic effect of the product combination, lower doses are assessed to be sure not to saturate thrombin detection.

The tested compositions contain:

| Combination | Hemlibra ® (μg/mL) | NovoSeven ® (μg/mL) |
|---|---|---|
| Combination 1 | 50 | 6 |
| Combination 2 | 50 | 5 |
| Combination 3 | 40 | 4 |
| Combination 4 | 30 | 3 |
| Combination 5 | 20 | 2 |
| Combination 6 | 10 | 1 |

Measuring the thrombogenic potential of the Sevenfact®+Hemlibra® combination after induction of coagulation with TF/PL:

The thrombin generation test is performed in 80 μL of an FVIII-deficient plasma pool that mimics a hemophilia A plasma in the presence of 20 μL of PPP reagent (Stago) containing 0.5 pM of tissue factor (TF) and 4 pM of phospholipids (PL).

The reaction is initiated by the addition of 20 μL of Fluca Kit (substrate+CaCl₂)) which is the start of the measurement of thrombin generation.

The thrombin generation test is conducted in the presence of 0.5 pM TF/4 pM PL (coagulation inducer) in several Sevenfact®/Hemlibra® combinations. The composition containing the largest quantity of product is made up of 6 μg/mL of Sevenfact® and 50 μg/mL of Hemlibra®, at their maximum.

The thrombogenic potential obtained in the presence of the combination of products is compared to the potential of the single products. To consider a synergistic effect of the product combination, lower doses are assessed to be sure not to saturate thrombin detection.

The tested compositions contain:

| Combination | Hemlibra ® (μg/mL) | Sevenfact ® (μg/mL) |
|---|---|---|
| Combination 1 | 50 | 6 |
| Combination 2 | 50 | 5 |
| Combination 3 | 40 | 4 |
| Combination 4 | 30 | 3 |
| Combination 4 | 20 | 2 |
| Combination 6 | 10 | 1 |

Measuring the thrombogenic potential of the Sevenfact®+Hemlibra® combination after induction of coagulation with cephalin:

The thrombin generation test is performed in 80 μL of an FVIII-deficient plasma pool that mimics a hemophilia A plasma in the presence of 20 μL of cephalin (CK-Prest reconstituted with 5 mL of distilled H₂O).

The reaction is initiated by the addition of 20 μL of Fluca Kit (substrate+CaCl₂)) which is the start of the measurement of thrombin generation.

The thrombin generation test is conducted in the presence of 20 μL of cephalin in several Sevenfact®/Hemlibra® combinations. The composition containing the largest quantity of product is made up of 6 μg/mL of Sevenfact® and 50 μg/mL of Hemlibra®, at their maximum.

The thrombogenic potential obtained in the presence of the combination of products is compared to the potential of the single products. To consider a synergistic effect of the product combination, lower doses are assessed to be sure not to saturate thrombin detection.

The tested compositions contain:

| Combination | Hemlibra ® (μg/mL) | Sevenfact ® (μg/mL) |
|---|---|---|
| Combination 1 | 50 | 6 |
| Combination 2 | 50 | 5 |
| Combination 3 | 40 | 4 |
| Combination 4 | 30 | 3 |
| Combination 5 | 20 | 2 |
| Combination 6 | 10 | 1 |

For all of these tests, the appearance of fluorescence is measured on a Fluoroskan Ascent fluorometer (ThermoLabsystems) at an excitation wavelength of 390 nm and an emission wavelength of 460 nm. Thrombinograms (curves showing the intensity of fluorescence over time) are then analyzed by using Thrombinoscope™ software, which converts the fluorescence value into nM of thrombin by a comparative calculation.

A synergistic effect is considered, for example, when at least one of the parameters calculated from the thrombin generation test for a given combination is greater than the sum of each of these parameters obtained with the components alone, deducted from the background noise of the experiment

### Example 4: Comparison of the Potential of Sevenfact™, Hemlibra® and the Combination of the Two in Hemophilia A Plasma

### Reagents:

thrombin calibrator (Stago)
1 pM TF PRP reagent (Stago)
4 μM PL MP reagent(Stago)
Fluo-buffer (Stago)
Fluo-substrate (Stago)
Sevenfact™: Transgenic factor VII produced in rabbits 1 mg/ml (LFB)
Hemlibra®: Emicizumab (Roche/Genentech/Chugaï)
Hemophilia A plasma (Cryopep)
Owren Koller (Stago)

### Method:

The thrombin generation test consists of activating coagulation ex vivo, for example with a mixture of tissue factor and phospholipids (TF/PL), then measuring the concentration of thrombin generated over time. The thrombin generation tests are conducted with 80 μL of hemophilia A plasma (Cryopep), in the presence of 20 μL of a mixture of PRP and MP reagents (Stago) containing 0.5 pM of tissue factor and 4 pM of phospholipids.

The reaction is initiated by the addition of 20 μL of Fluca Kit (Fluo substrate+CaCl₂)) which is the start of the measurement of thrombin generation (TG).

Fluorescence is measured by fluorimetry using the Fluoroskan Ascent device (ThermoLabsystems) at an excitation wavelength of 390 nm and an emission wavelength of 460 nm. The thrombinograms are analyzed with Thrombinoscope™ software that uses a comparative calculation to convert fluorescence intensity into molar concentration of thrombin (nM).

To measure the thrombogenic potential of the two molecules, several hemophilia A plasmas are studied. The highest therapeutic dose of FVIIa is 270 μg/kg, which corresponds to 6 μg/mL of FVIIa (or 120 nM) in the plasma. The use of this dose can be considered as a maximum potential for thrombin generation. On the basis of the product concentrations in the bloodstream obtained in patients, Sevenfact™ concentrations comprised 20 and 100 nM are also studied. Hemlibra® (Roche/Genentech/Chugaï, USA), a bispecific antibody imitating the function of FVIII, is used at a maximum concentration of 120 μg/mL. The concentration actually detected in patients on treatment is 50 μg/mL (or 300 nM) (Oldenburg et al. NEJM, 2017). Thus, Hemlibra® is used here at approximately 300 nM (50 μg/mL). The variables studied to measure the thrombogenic potential of Hemlibra® and Sevenfact™ are:
the endogenous thrombin potential (ETP): area under the curve representing the total quantity of thrombin generated,
peak height: maximum concentration of thrombin measured, and
thrombin generation velocity: the thrombin formation speed.

### 2—Results

### 2.1—Effect of Sevenfact™ or Hemlibra® on Hemophilia A Plasmas

### 2.1.1—Assessment in Batch 1 of Hemophilia A Plasma

In this matrix, very low thrombin generation signals coming from the two compounds are obtained, regardless of the concentrations used. Indeed, the thrombin generation observed is almost zero for Hemlibra® and Sevenfact™ at concentrations of 20 and 40 nM. With 100 nM of Sevenfact™, a very low thrombin generation peak is observed (Table 1).

TABLE 1
Thrombin generation parameters from batch 1 of Hemophilia A plasma
treated with Sevenfact ™ or Hemlibra ®

| | 20 nM | 40 nM | 100 nM | 300 nM |
|---|---|---|---|---|
| | Sevenfact ™ | Sevenfact ™ | Sevenfact ™ | Hemlibra ® |
| ETP | 125 | 154.25 | 128.75 | 128.25 |
| (nM · min) | | | | |
| Peak (nM) | 7.105 | 9.46 | 16.55 | 6.41 |
| Velocity | 0.59 | 0.84 | 1.64 | 0.42 |
| (nM/min) | | | | |

Thus, each molecule used individually only induces a very low generation of thrombin.

### 2.1.2—Assessment in Batch 2 of Hemophilia A Plasma

A second batch of Hemophilia A plasma was tested. There again, a very low generation of thrombin is observed with the use of Hemlibra® and Sevenfact™, with a maximum thrombin generation peak at a concentration of 100 nM of Sevenfact™ (Table 2).

TABLE 2
Thrombin generation parameters from batch 2 of Hemophilia A plasma
treated with Sevenfact ™ or Hemlibra ®

| | 20 nM | 40 nM | 100 nM | 300 nM |
|---|---|---|---|---|
| | Sevenfact ™ | Sevenfact ™ | Sevenfact ™ | Hemlibra ® |
| ETP | 221.25 | 280.75 | 414.25 | 196 |
| (nM · min) | | | | |
| Peak (nM) | 9.93 | 12.84 | 21.04 | 8.895 |
| Velocity | 0.7 | 0.96 | 1.75 | 0.51 |
| (nM/min) | | | | |

In this matrix, Sevenfact™ and Hemlibra® used separately have a low thrombogenic potential.

### Example 5: Assessment of the Synergistic Combination of Sevenfact™+Hemlibra®

### 1—Protocol

The reagents, device and experimental protocol in hemophilia A plasma are identical to those described in Example 2.

### 2—Results

As seen in Example 2, Sevenfact™ and Hemlibra® used individually induce a low thrombin generation in Hemophilia A plasma. The synergistic effect of the Sevenfact™ and Hemlibra® combination is studied here. Three concentrations of Sevenfact™ are studied (20 nM, 40 nM and 100 nM) in the presence of a Hemlibra® concentration of 300 nM. A synergistic effect is taken into account if the effect of the Sevenfact™+Hemlibra® combination is at least 2 times greater than the sum of the effects of Sevenfact and Hemlibra® taken separately for at least one of the parameters of the thrombin generation test (ETP, peak thrombin generation and velocity).

2.1—Effect of Sevenfact™ and Hemlibra® on Hemophilia A Plasmas after Coagulation Induction with TF/PL

2.1.1—Assessment in Batch 1 of Hemophilia A Plasma

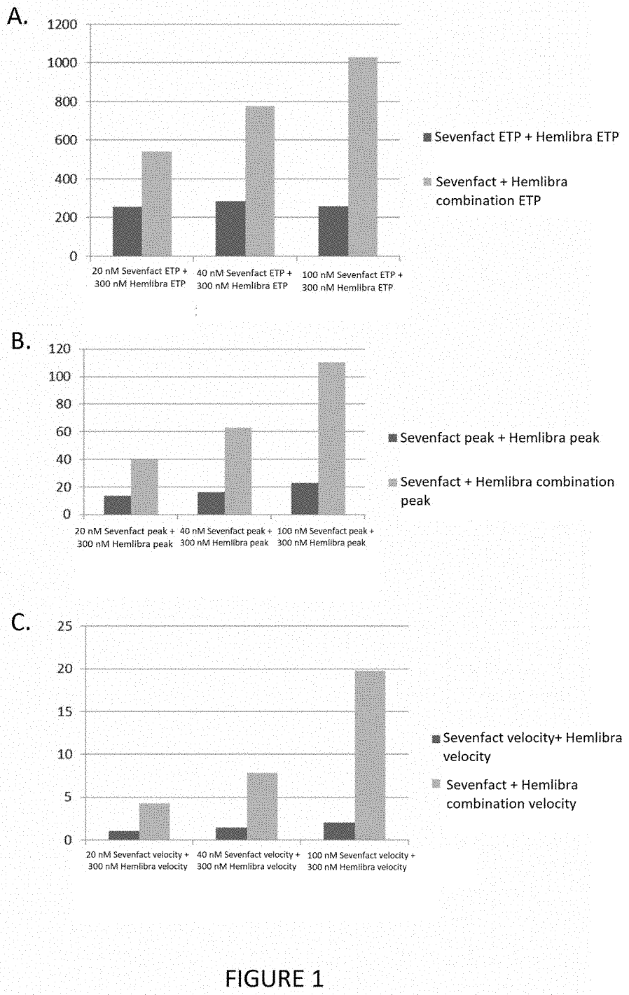

The results are shown in Table 3 and FIG. 1. At a very low Sevenfact™ concentration of 20 nM, the ratios for ETP (FIG. 1A), thrombin peak (FIG. 1B) and velocity (FIG. 10) of the Sevenfact™+Hemlibra® combination are, respectively, 2.14, 2.95 and 4.19. Thus, even at the lowest concentration tested, a synergistic thrombogenic effect is observed.

At a concentration of 40 nM, for all the parameters tested, the ratio is greater than 2. The ratio obtained for ETP is 2.75 (FIG. 1A), the ratio obtained for thrombin peak is 3.96 (FIG. 1B) and the one obtained for velocity reaches a value of 6.21 (FIG. 10). In other words, the thrombin formation speed is six times higher when Sevenfact™ and Hemlibra® are used in combination

The synergistic effect is the greatest at a Sevenfact™ concentration of 100 nM. At a concentration of 100 nM, for all the parameters tested, the ratio is greater than 2. The ratio obtained for ETP is 4.00 (FIG. 1A) and the ratio for thrombin peak is 4.81 (FIG. 1B), which means that the maximum concentration of thrombin generated is almost five times greater when Hemlibra® and Sevenfact™ are used in combination. The corresponding velocity ratio is 9.58 (FIG. 10), which means that thrombin is generated almost ten times faster when Sevenfact™ and Hemlibra® are used in combination.

TABLE 3
Thrombin generation parameters from batch 1 of Hemophilia A plasma treated with the Sevenfact ™ + Hemlibra ® combination

| 20 nM | 40 nM | 100 nM | Combinations | | | | | |
|---|---|---|---|---|---|---|---|---|
| Sevenfact ™ | Sevenfact ™ | Sevenfact ™ | ETP | ETP | ETP | | | |
| ETP + | ETP + | ETP + | (20 nM | (40 nM | (100 nM | Combination/sum ratios | | |
| 300 nM | 300 nM | 300 nM | Sevenfact ™ + | Sevenfact ™ + | Sevenfact ™ + | 20 | 40 | 100 |
| Hemlibra ® | Hemlibra ® | Hemlibra ® | 300 nM | 300 nM | 300 nM | nM | nM | nM |
| ETP | ETP | ETP | Hemlibra ®) | Hemlibra ®) | Hemlibra ®) | Sevenfact ™ | Sevenfact ™ | Sevenfact ™ |
| 253.25 | 282.50 | 257 | 543 | 777.50 | 1029 | 2.14 | 2.75 | 4.00 |
| 20 nM | 40 nM | 100 nM | Combinations | | | | | |
| Sevenfact ™ | Sevenfact ™ | Sevenfact ™ | Peak | Peak | Peak | | | |
| peak + | peak + | peak + | (20 nM | (40 nM | (100 nM | Combination/sum ratios | | |
| 300 nM | 300 nM | 300 nM | Sevenfact ™ + | Sevenfact ™ + | Sevenfact ™ + | 20 | 40 | 100 |
| Hemlibra ® | Hemlibra ® | Hemlibra ® | 300 nM | 300 nM | 300 nM | nM | nM | nM |
| peak | peak | peak | Hemlibra ®) | Hemlibra ®) | Hemlibra ®) | Sevenfact ™ | Sevenfact ™ | Sevenfact ™ |
| 13.515 | 15.87 | 22.965 | 39.915 | 62.9 | 110.565 | 2.95 | 3.96 | 4.81 |
| 20 nM | 40 nM | 100 nM | Combinations | | | | | |
| Sevenfact ™ | Sevenfact ™ | Sevenfact ™ | Velocity | Velocity | Velocity | | | |
| velocity + | velocity + | velocity + | (20 nM | (40 nM | (100 nM | Combination/sum ratios | | |
| 300 nM | 300 nM | 300 nM | Sevenfact ™ + | Sevenfact ™ + | Sevenfact ™ + | 20 | 40 | 100 |
| Hemlibra ® | Hemlibra ® | Hemlibra ® | 300 nM | 300 nM | 300 nM | nM | nM | nM |
| velocity | velocity | velocity | Hemlibra ®) | Hemlibra ®) | Hemlibra ®) | Sevenfact ™ | Sevenfact ™ | Sevenfact ™ |
| 1.01 | 1.42 | 2.06 | 4.24 | 7.86 | 19.78 | 4.19 | 6.21 | 9.58 |

In conclusion, for all the Sevenfact™ concentrations tested, Sevenfact™ and Hemlibra® used in combination have a synergistic effect on thrombin generation.

2.1.2—Assessment in Batch 2 of Hemophilia A Plasma

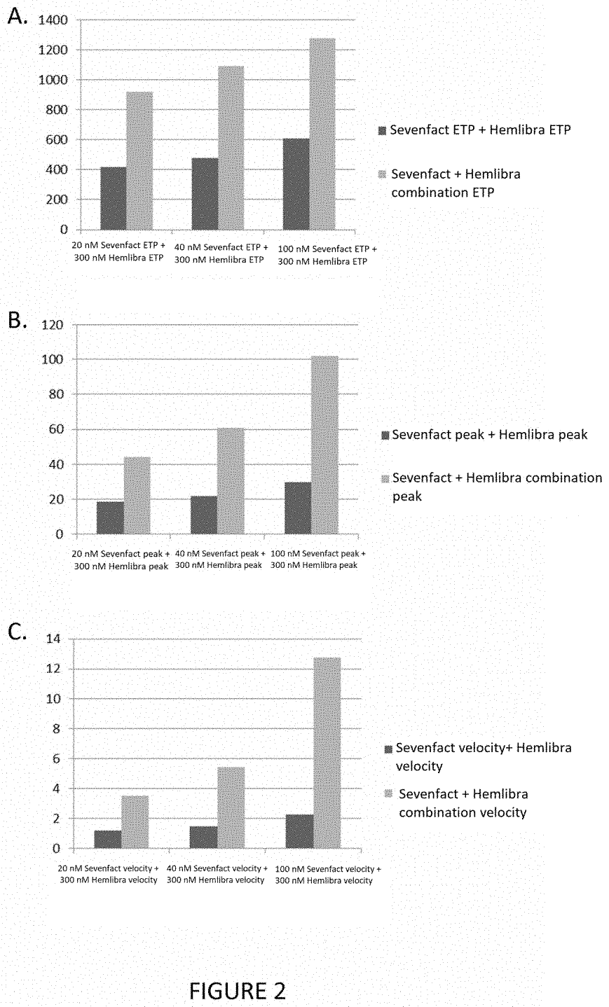

The results are shown in Table 4 and FIG. 2. At a very low Sevenfact™ concentration of 20 nM, a ratio of 2.21 is obtained for the ETP parameter (FIG. 2A), a ratio of 2.34 is obtained for the thrombin peak (FIG. 2B), and a ratio of 2.9 is obtained for the velocity parameter (FIG. 2C) of the Sevenfact™+Hemlibra® combination. Thus, even at the lowest Sevenfact™ concentration tested, a synergistic thrombogenic effect is observed.

At a concentration of 40 nM, the ratio corresponding to ETP is 2.29 (FIG. 2A), that corresponding to thrombin peak is 2.79 (FIG. 2B) and the ratio corresponding to velocity is 3.68 (FIG. 2C), which means that the use of Sevenfact™ in combination with Hemlibra® enables thrombin to be formed approximately 4 times faster.

The synergistic effect is the greatest with a Sevenfact™ concentration of 100 nM. At a concentration of 100 nM, the ratio corresponding to the thrombin generation peak is 3.41 (FIG. 2B) and that corresponding to velocity is 5.63 (FIG. 2C), which means that thrombin is generated almost 6 times faster and that the thrombin concentration achieved is almost four times greater when Sevenfact™ is used in combination with Hemlibra®.

TABLE 4
Thrombin generation parameters from batch 2 of Hemophilia A plasma treated with the Sevenfact ™ + Hemlibra ® combination

| 20 nM | 40 nM | 100 nM | Combinations | | | | | |
|---|---|---|---|---|---|---|---|---|
| Sevenfact ™ | Sevenfact ™ | Sevenfact ™ | ETP | ETP | ETP | | | |
| ETP + | ETP + | ETP + | (20 nM | (40 nM | (100 nM | Combination/sum ratios | | |
| 300 nM | 300 nM | 300 nM | Sevenfact ™ + | Sevenfact ™ + | Sevenfact ™ + | 20 | 40 | 100 |
| Hemlibra ® | Hemlibra ® | Hemlibra ® | 300 nM | 300 nM | 300 nM | nM | nM | nM |
| ETP | ETP | ETP | Hemlibra ®) | Hemlibra ®) | Hemlibra ®) | Sevenfact ™ | Sevenfact ™ | Sevenfact ™ |
| 417.25 | 476.75 | 610.25 | 920.75 | 1091.25 | 1275.50 | 2.21 | 2.29 | 2.09 |
| 20 nM | 40 nM | 100 nM | Combinations | | | | | |
| Sevenfact ™ | Sevenfact ™ | Sevenfact ™ | Peak | Peak | Peak | | | |
| peak + | peak + | peak + | (20 nM | (40 nM | (100 nM | Combination/sum ratios | | |
| 300 nM | 300 nM | 300 nM | Sevenfact ™ + | Sevenfact ™ + | Sevenfact ™ + | 20 | 40 | 100 |
| Hemlibra ® | Hemlibra ® | Hemlibra ® | 300 nM | 300 nM | 300 nM | nM | nM | nM |
| peak | peak | peak | Hemlibra ®) | Hemlibra ®) | Hemlibra ®) | Sevenfact ™ | Sevenfact ™ | Sevenfact ™ |
| 18.825 | 21.735 | 29.935 | 43.96 | 60.745 | 101.975 | 2.34 | 2.79 | 3.41 |
| 20 nM | 40 nM | 100 nM | Combinations | | | | | |
| Sevenfact ™ | Sevenfact ™ | Sevenfact ™ | Velocity | Velocity | Velocity | | | |
| velocity + | velocity + | velocity + | (20 nM | (40 nM | (100 nM | Combination/sum ratios | | |
| 300 nM | 300 nM | 300 nM | Sevenfact ™ + | Sevenfact ™ + | Sevenfact ™ + | 20 | 40 | 100 |
| Hemlibra ® | Hemlibra ® | Hemlibra ® | 300 nM | 300 nM | 300 nM | nM | nM | nM |
| velocity | velocity | ETP | Hemlibra ®) | Hemlibra ®) | Hemlibra ®) | Sevenfact ™ | Sevenfact ™ | Sevenfact ™ |
| 1.21 | 1.48 | 2.27 | 3.52 | 5.44 | 12.77 | 2.9 | 3.68 | 5.63 |

In conclusion, for all the Sevenfact™ concentrations tested, Sevenfact™ and Hemlibra® used in combination have a synergistic effect on thrombin generation.

1. Pharmaceutical composition comprising:
a. transgenic factor VII, and
b. a multispecific antibody directed against factor IX and factor X,

2. Pharmaceutical composition according to claim 1, wherein said transgenic factor VII is a human factor VII derived from production by epithelial cells of the mammary glands of a transgenic non-human mammal.

3. Pharmaceutical composition according to claim 2, wherein said transgenic mammal is a rabbit.

4. Pharmaceutical composition according to any one of claims 1 to 3, wherein the antibody is emicizumab.

5. Combination product comprising:
a. transgenic factor VII, and
b. a multispecific antibody directed against factor IX and factor X,
for its use in preventing or treating a coagulation disorder in a patient.

6. Combination product according to claim 5, in the treatment of hemophilia A.

7. Combination product according to one of claim 5 or 6 in the treatment of hemophilia A with factor VIII inhibitors.

8. Combination product for its use according to claims 5 to 7, said combination product being in the form of a pharmaceutical composition such as defined in any one of claims 1 to 4.

9. Combination product for its use according to claims 5 to 8, said factor Vila and said antibody being in a form suited to simultaneous administration to the patient.

10. Combination product for its use according to claims 5 to 8, said factor Vila and said antibody being in forms suited to separate administration to the patient.

11. Kit comprising
A container containing transgenic factor FVII; and
Another container containing an antibody directed against factor IX and factor X.

12. Method to treat a coagulation disorder in a patient, which method comprises the simultaneous or sequential administration to said patient of transgenic factor VII and a multispecific antibody directed against factor IX and factor X.

13. Use of a combination of transgenic factor VII and a multispecific antibody directed against factor IX and factor X for the treatment of a coagulation disorder in a patient, preferably hemophilia A, with factor VIII inhibitors.
